(19) United States
(12) Patent Application Publication
(10) Pub. No.: US 2007/0268995 A1
(43) Pub. Date: Nov. 22, 2007

(54) CT SCANNER WITH HELICAL PATH SOURCE

(76) Inventors: Joseph Webster Stayman, Ann Arbor, MI (US); Predrag Sukovic, Birmingham, MI (US); Neal Clinthorne, Ann Arbor, MI (US); Miodrag Rakic, Redondo Beach, CA (US)

Correspondence Address: CARLSON, GASKEY & OLDS, P.C., 400 WEST MAPLE ROAD, SUITE 350, BIRMINGHAM, MI, 48009, US

(21) Appl. No.: 11/744,430

(22) Filed: May 4, 2007

Related U.S. Application Data
(60) Provisional application No. 60/797,538, filed on May 4, 2006.

(51) Int. Cl. H05G 1/60; G21K 5/10; A61B 6/00; G01N 23/00; G21K 1/12

(52) U.S. Cl. 378/4; 378/146

(57) ABSTRACT

An example CT scanner assembly includes a gantry having a first end and a second end rotatable about a first axis, an x-ray detector adjacent the first end, and an x-ray source adjacent the second end. The x-ray source directs an x-ray beam toward a portion of the x-ray detector. The x-ray source translates along a second axis aligned with the first axis when the gantry rotates.

This application claims priority to U.S. Provisional Application No. 60/797538 filed May 4, 2006.

### BACKGROUND OF THE INVENTION

The present invention relates generally to computer tomography (CT) scanners and more particularly to a CT scanner which collimates an x-ray to assist in analysis of x-ray scatter during a helical-type scan.

Generally, CT scanners include a moveable ring with an x-ray source mounted on the movable ring and an x-ray detector mounted opposite the x-ray source. The patient lies on a platform that moves through the ring. The ring is rotated so the x-ray source and the x-ray detector revolve in a helical path around the patient while taking a series of x-rays. The x-ray source produces an x-ray signal that is directed toward the x-ray detector. The x-ray source and x-ray detector typically translate together when following the helical path.

The x-ray signal scatters as it travels the distance between the x-ray source and the x-ray detector. The scatter alters the image that is created from the x-ray signal and hinders the doctor when analyzing the CT image. When the effect of the scatter is known, it can be filtered from the received signal to provide a more accurate image.

Therefore, it is desirable to provide a CT scanner which supplies information on the scatter of an x-ray signal for use in analyzing the x-ray image.

### SUMMARY OF THE INVENTION

An example CT scanner assembly includes a gantry having a first end and a second end rotatable about a first axis, an x-ray detector adjacent the first end, and an x-ray source adjacent the second end. The x-ray source directs an x-ray beam toward a portion of the x-ray detector. The x-ray source translates along a second axis aligned with the first axis when the gantry rotates.

Another example CT scanner assembly includes a gantry having an x-ray source and an x-ray detector rotatable about a patient to define an axis, and an x-ray beam moves from the x-ray source to the x-ray detector. The CT scanner includes at least one shield for collimating a portion of the x-ray beam and a computer for identifying scatter in an x-ray image using known scatter from a collimated portion of the x-ray beam.

An example method for generating a CT image include the steps of: (a) rotating an x-ray source and an x-ray detector about a patient, (b) taking a plurality of x-ray images with the x-ray source and the x-ray detector during step (a), and (c) translating the x-ray source vertically during step (a).

### BRIEF DESCRIPTION OF THE DRAWINGS

These and other features of the present invention can be best understood from the following specification and drawings, the following of which is a brief description.

FIG. 1 illustrates an example CT scanner having a gantry.

FIG. 2 illustrates another example CT scanner having a gantry.

FIG. 3 illustrates an example image from the CT scanner.

### DETAILED DESCRIPTION OF THE PREFERRED EMBODIMENT

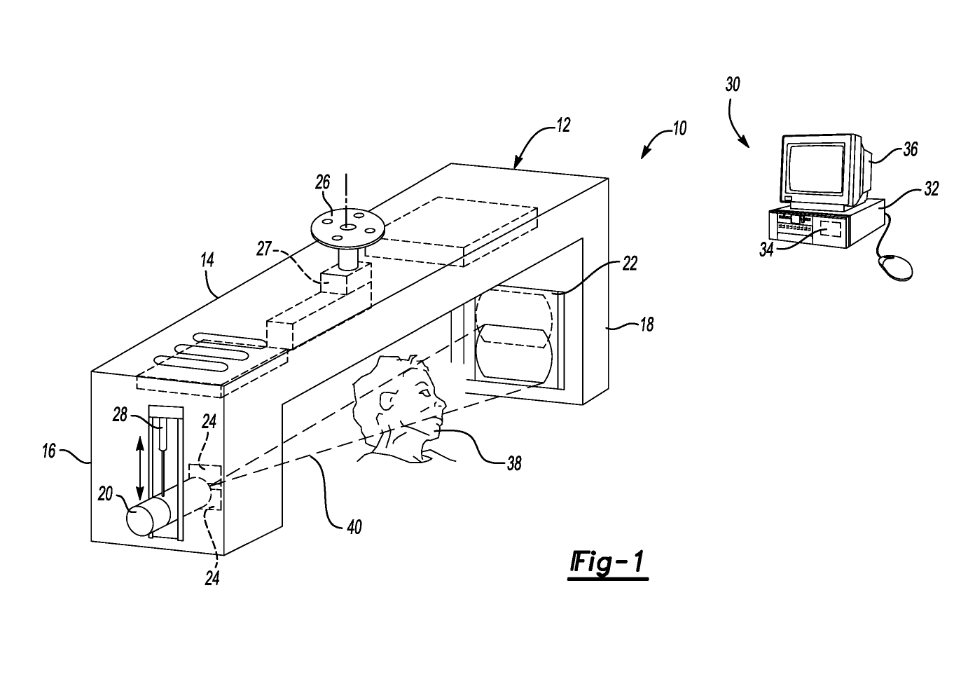

FIG. 1 illustrates a CT scanner 10 according to the present invention wherein all of the components are contained in a gantry 12. The gantry 12 provides the structural support and housing for the components. The gantry 12 comprises a cross-bar section 14 from which a first arm 16 and a second arm 18 extend perpendicularly from either end, forming a c-shaped assembly.

The first arm 16 houses an x-ray source 20 that generates x-rays 40, which in this embodiment is a cone-beam x-ray source. The second arm 18 houses a complementary x-ray detector 22. The cross-bar section 14 of the gantry 12 houses a motor 27 for rotating the gantry 12 relative to a mounting plate 26. Alternatively, the motor 27 could be mounted off the gantry 12. A pair of shields 24 are supported on the x-ray source 20 to collimate the x-rays 40. In the embodiment shown, the pair of shields 24 block at least an upper and lower portion of the x-ray source 20, preventing x-rays 40 from passing through the pair of shields 24. Only the middle portion of the x-ray 40 passes through and is received by the x-ray detector 22. Although not shown, the x-ray shields 24 would preferably block a portion of each side of the x-ray source 20, allowing the middle portion of the x-ray 40 to pass through.

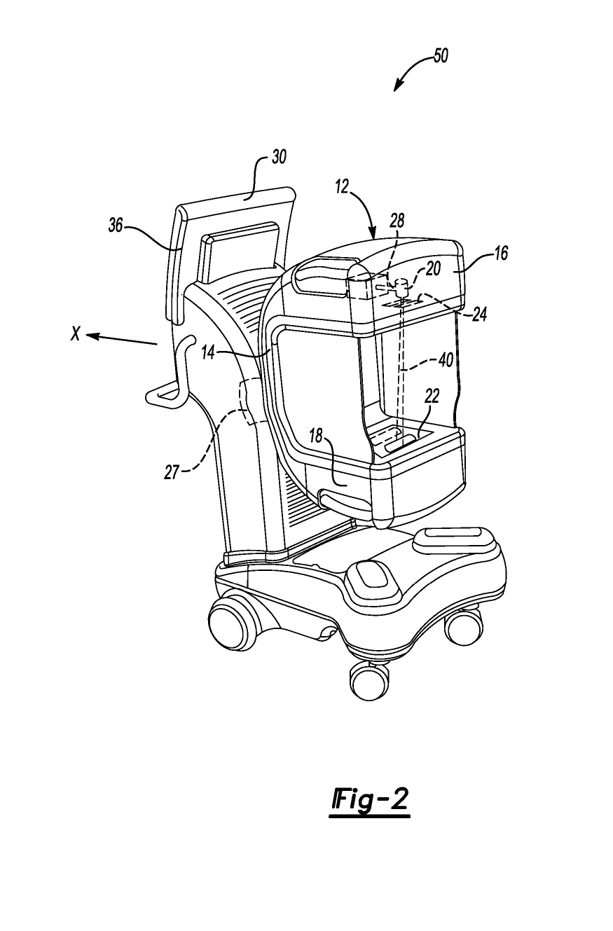

The first arm 18 further includes an actuator 28 for moving the x-ray source 20 during scanning. In this example, the x-ray source 20 moves vertically during scanning in a direction aligned with, and substantially parallel to, the axis of rotation of the gantry 12. In another example CT scanner, the x-ray source 20 moves horizontally as the gantry 12 rotates about a horizontal axis, as shown in an example scanner 50 of FIG. 2.

Referring back to FIG. 1, the x-ray 40 shown projected on the x-ray detector 22 is moved from an upper portion of the x-ray detector 22 to a lower portion of the x-ray detector 22 (or vice versa) during scanning, as shown. The x-ray detector 22 is larger than the portion of the x-ray 40 striking the x-ray detector 22 directly, as is also shown.

The example CT scanner 10 may further include a computer 30 including a microprocessor or CPU 32, memory 34, a monitor 36 and other hardware and software for performing the functions described herein. The computer 30 controls the rotation of the CT scanner 10, the location and operation of the x-ray source 20 and the x-ray detector 22, and collects the data from the x-ray detector 22 and stores it for later collection, such as in memory 34, hard drive, optical, magnetic or other storage. The computer 30 could also be mounted on-board the gantry 12.

In operation, a part of the body, such as a head 38, is positioned between the first arm 16 and the second arm 18 of the gantry 12. The computer 30 powers the x-ray source 20. The x-ray source 20 generates an x-ray 40 that is directed toward the x-ray detector 22. The CPU 32 then controls the motor 27 to perform one complete revolution of the gantry 12, during which time the computer 30 collects multiple images from the x-ray detector 22. During the revolution, the actuator 28 moves the x-ray source 20 relative to the x-ray detector 22, such that the x-ray source 20 follows a helical path to cover a larger portion of the patient during the revolution and to eliminate some cone-beam artifacts. The images taken by the x-ray detector 22 are stored in the storage 34.

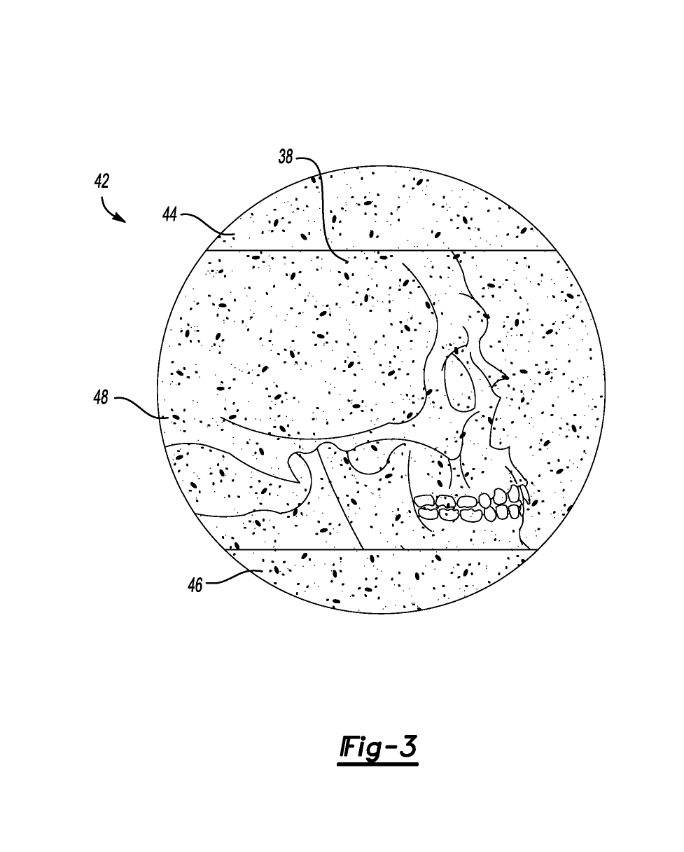

An image 42 taken from the example collimated CT scanner 10 is shown in FIG. 3. An upper portion 44 and a lower portion 46 of the image 42 represent the portion of the x-ray 40 that is collimated by the pair of x-ray shields 24. A central uncollimated portion 48 includes an image of the head 38.

The image 42 includes randomly distributed scatter. The upper portion 44 and the lower portion 46 represent only scatter, while the central portion 48 includes scatter and the image of the head 38. Based upon the known scatter information provided in the upper portion 44 and the lower portion 46 of the image 42, the computer 30 can predict the effect of the scatter on the image 42 and specifically the image of the head 38. A person having ordinary skill in the art would be able to develop a suitable technique for predicting the effect of scatter on an image if provided known scatter information. The computer 30 uses the predicted effect of scatter to improve the image 42 by removing or reducing the effect of scatter in the image 42.

Although a preferred embodiment of this invention has been disclosed, a worker of ordinary skill in this art would recognize that certain modifications would come within the scope of this invention. For that reason, the following claims should be studied to determine the true scope and content of this invention.

1. A CT scanner assembly, comprising:
a gantry having a first end and a second end, wherein said gantry is rotatable about a first axis;
an x-ray detector adjacent said first end; and
an x-ray source adjacent said second end for directing an x-ray beam toward a portion of said detector, wherein said x-ray source translates relative to the x-ray detector along a second axis aligned with said first axis when said gantry rotates.

2. The CT scanner assembly of claim 1, including at least one shield for collimating a portion of said x-ray beam.

3. The CT scanner assembly of claim 2, including a computer for capturing an x-ray image from said x-ray detector, said x-ray image including a collimated portion, wherein said computer predicts scatter in said x-ray image using known scatter from said collimated portion.

4. The CT scanner assembly of claim 2, wherein said at least one shield comprises a top shield and a bottom shield.

5. The CT scanner assembly of claim 1, wherein said x-ray beam contacts different portions of said x-ray detector as said x-ray source translates.

6. The CT scanner assembly of claim 1, wherein said x-ray detector is larger than a portion of said x-ray beam striking said x-ray detector.

7. The CT scanner assembly of claim 1, wherein said second axis is substantially parallel to said first axis.

8. A CT scanner assembly, comprising:
a gantry having an x-ray source and an x-ray detector, wherein said gantry is rotatable about an axis about a patient;
an x-ray beam directed from said x-ray source to said x-ray detector;
at least one shield for collimating a portion of said x-ray beam; and
a computer for identifying scatter in an x-ray image using known scatter from a shielded portion of said x-ray image.

9. The CT scanner assembly of claim 8, wherein said x-ray source translates in a direction substantially aligned with said axis as said gantry rotates.

10. The CT scanner assembly of claim 9, wherein said direction is substantially parallel to said axis.

11. The CT scanner assembly of claim 8, wherein said at least one shield comprises an upper shield and a lower shield that collimates an upper portion and a lower portion, respectively, of said x-ray beam.

12. The CT scanner assembly of claim 8, wherein said x-ray beam is a cone beam x-ray.

13. A method for generating a CT image, the method comprising the steps of:
a) rotating an x-ray source and an x-ray detector about a patient to define a first axis;
b) taking a plurality of x-ray images with the x-ray source and the x-ray detector during said step a); and
c) translating the x-ray source relative to the x-ray detector during said step a) along a second axis aligned with said first axis.

14. The method of claim 13, including the step of collimating an x-ray beam from the x-ray source.

15. The method of claim 13, including the step of collimating an upper portion and a lower portion of one of the plurality of x-ray images.

16. The method of claim 15, including the step of predicting scatter in the plurality of x-ray images using the upper portion and the lower portion.

17. The method of claim 16, including the step of improving one of the plurality of x-ray images using predicted scatter.
